(19) United States
(12) Patent Application Publication
(10) Pub. No.: US 2008/0233055 A1
(43) Pub. Date: Sep. 25, 2008

(54) FLAVORED ORAL CARE STRIP

(75) Inventors: Jeffrey J. Fisher, Ada, MI (US); Jack M. Frick, Hudsonville, MI (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: RANIR, LLC, Grand Rapids, MI (US)

(21) Appl. No.: 11/687,948

(22) Filed: Mar. 19, 2007

(51) Int. Cl. A61K 8/04

(52) U.S. Cl. 424/49

(57) ABSTRACT

An oral care strip includes a substrate having a first side and a second side. An oral care substance is disposed on the first side and a flavor element is attached to the second side. The flavor element may be a dissolvable, edible, flavored film. A method for forming the oral care strip includes the steps of a) applying an oral care substance to a first side of a substrate and b) attaching a flavor element to a second side of the substrate. The second side of the substrate may include a wax layer to which the flavor element is attached.

### BACKGROUND OF THE INVENTION

The present invention relates generally to oral care strips, and more particularly to a flavored oral care strip and a method of making the same.

Oral care strips have recently grown in popularity as an effective way to deliver an oral care substance, such as a tooth whitening agent, to a tooth or multiple teeth. In one known embodiment, the strip includes a thin fabric substrate having a size sufficient to cover multiple teeth. The substrate has a tooth whitening agent on one side and a wax on the other side. The tooth whitening agent is covered by a releasable carrier, such as a mylar film. In use, the carrier is peeled away by a consumer to expose the whitening agent, and the strip is placed in the consumer's mouth with the whitening agent in contact with the front surface of the consumer's teeth. A portion of the strip is folded behind the teeth so the strip can be pinched together about the teeth. The wax layer prevents the strip from sticking to the inside of the mouth, and helps to keep the strip formed about the teeth. The strip is left in the consumer's mouth a predetermined length of time such that the agent can act to whiten the teeth.

Unfortunately, while oral care strips provide the benefit of whiter teeth, there are some difficulties in using them, primarily in the lack of comfort and enjoyment. Many of the strips can be uncomfortable to keep in the mouth for a prolonged period, and the whitening gel often has a displeasing taste. In an attempt to overcome these difficulties, some manufacturers have added flavoring agents to the whitening agent; however, when combined, these agents can react with each other and become unstable. In another case, shown in U.S. Pat. No. 6,916,463 to Lee, a flavoring agent is added to the side of the substrate opposite the whitening agent by chemically linking the flavoring agent to a phosphate and then adhering the composition to the substrate with a carrier material. However, manufacturers are continually searching for ways to more effectively overcome these difficulties.

### SUMMARY OF THE INVENTION

The aforementioned problems are overcome by the present invention, wherein an oral care strip is provided with a flavor element.

In one aspect, the oral care strip includes a substrate having a first side and a second side. An oral care substance, such as a tooth whitening agent, is disposed on the first side and covered with a releasable protective carrier. A flavor element is attached to the second side opposite the oral care substance, so that the flavor element is exposed to the interior of a user's mouth when the oral care substance is placed in contact with the user's teeth. The flavor element may be a dissolvable, edible, flavored film, such as the breath strips that are known for placement on a user's tongue to enhance breath odor. In one embodiment, the flavor element may be a plurality of ground-up particles of the dissolvable edible film left over from the production of breath strips. The second side of the substrate may include wax, and the particles may be attached to the surface of the wax.

In a second aspect, the present invention includes a method for forming a flavored oral care strip. The method includes the steps of a) applying an oral care substance to a first side of a substrate and b) attaching a flavor element to a second side of the substrate. The second side of the substrate may additionally include a molten wax, and the flavor element may be placed onto a surface of the molten wax opposite the substrate, in which case the flavor element is retained in the wax as the wax solidifies. First and second removable carrier films may be placed over the oral care substance and over the flavor element.

The present invention makes the use of oral care strips more enjoyable by effectively adding flavoring to the strips opposite the oral care substance. In addition, the use of left over particles of dissolvable film is cost effective. Thirdly, the method of applying the particles to a molten wax enables the addition of flavoring without a significant loss of manufacturing time.

These and other objects, advantages, and features of the invention will be readily understood and appreciated by reference to the detailed description of the current embodiment and the drawings.

### BRIEF DESCRIPTION OF THE DRAWINGS

FIG. 1 is a schematic view of one embodiment of the method of manufacturing the present invention.

FIG. 2 is a top view of the oral care strip according to one embodiment.

FIG. 3 is a cross sectional view thereof along line 3-3 in FIG. 2.

FIG. 4 is an exploded view thereof.

FIG. 5 is a perspective view of the oral care strip as typically received by a consumer.

FIG. 6 is a perspective view of the oral care strip including both releasable carriers.

### DETAILED DESCRIPTION OF THE INVENTION

I. Overview

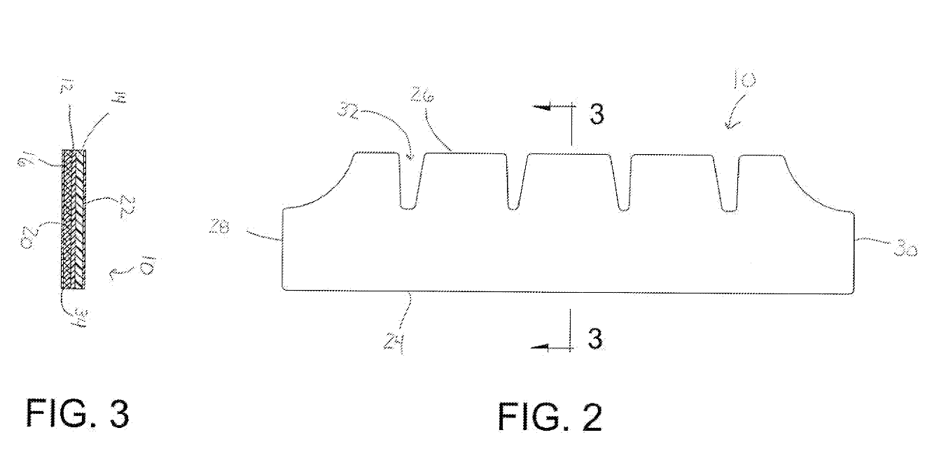

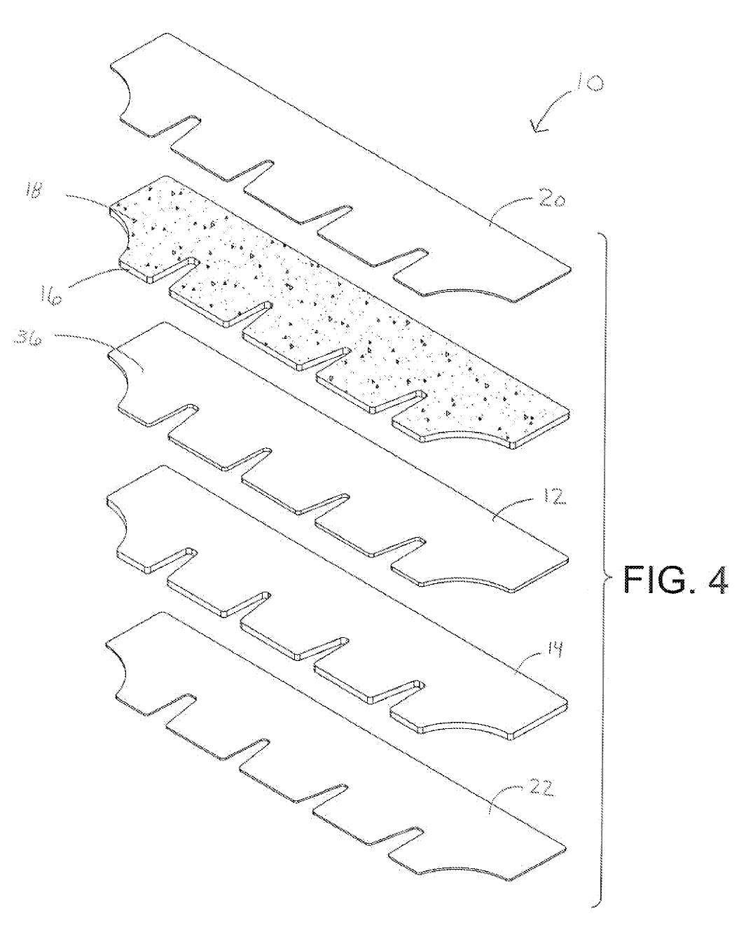

An oral care strip according to one embodiment of the present invention is shown in FIGS. 2-6 and generally designated 10. As shown in FIG. 4, the oral care strip 10 is a thin strip comprised of multiple layers, including a substrate 12, an oral care substance 14, a wax layer 16, a flavor element 18, a first releasable carrier 20 over the flavor element 18 and a second releasable carrier 22 over the oral care substance 14. In use, the strip 10 is inserted into a user's mouth such that the oral care substance 14 contacts at least the front surface of the teeth. A portion of the strip may be folded around the rear surfaces of the teeth to hold the strip on the teeth. The flavor element 18 is exposed to the user's mouth to add a desired flavor while the strip is in the user's mouth.

II. Structure

The substrate layer 12 is a thin, bendable strip comprised of one of a variety of materials. In one embodiment, the substrate 12 is a non-woven polyester. Alternatively, the substrate may be foam, a woven or non-woven fabric, or any other desired strip material that would not be harmful if placed in a person's mouth. As shown in FIG. 2, the strip 10 and substrate 12 are cut to fit over multiple teeth in a user's mouth. The strip includes an upper edge 24, a lower edge 26 and first and second side edges 28 and 30. The lower edge 26 may define a number of slits 32. The slits 32 enable the lower edge 26 to be folded easily about the rear surface of a user's teeth.

The oral care substance 14 is an agent that whitens or otherwise improves the appearance of teeth. Oral care substances are known in the art, and therefore will not be described in great detail. In one embodiment, the oral care substance is applied to the lower surface 34 of the substrate 12 to form a tooth contacting surface of the strip 10. The oral care substance 14 may be a homogeneous gel that is uniformly and continuously coated on the lower surface 34 of the substrate 12. Alternatively, the substance 14 may be a laminate or separated layers of components, an amorphous mixture of components, separate stripes of spots or other patterns of different components, or a combination of these structures. The substance 14 contains an active ingredient that is capable of influencing or effecting a desired change in appearance and/or structure of the surface it contacts. Example active ingredients include: hydrogen peroxide, carbamide peroxide, sodium fluoride, sodium monofluorophosphate, pyrophosphate, chlorhexidine, polyphosphate, triclosan and enzymes. Examples of appearance and structural changes include, but are not limited to, whitening, stain bleaching, stain removal, remineralization to form fluorapatite, plaque removal and tartar removal.

The wax layer 16 comprised of a wax or wax-like material and is generally applied or attached to the upper surface 36 of the substrate 12. This layer is located opposite the tooth contacting layer of oral care substance 14 such that it is exposed to the inside of the user's mouth when the tooth contacting layer contacts the surface of the user's teeth. The wax layer 16 helps prevent the strip 10 from sticking to the inside of the user's mouth, and provides a bendable substance that help form the strip 10 to the surface contour of the teeth. As an alternative to wax, the wax layer 16 may be formed from plastic, putty, or another deformable material.

The flavor element 18 may be one of a variety of known flavorings or flavored substances. In one embodiment, the flavor element 18 is comprised of a plurality of particles of a thin, edible, dissolvable, flavored film. The film may be comprised of a known dissolvable binder, such as pullulan, and a flavor ingredient such as mint, for example, peppermint, spearmint or wintergreen or other flavors such as cinnamon, berry or citrus. As illustrated, the flavor element 18 is disposed on the strip 10 opposite the tooth contacting oral care substance 14. In the illustrated embodiment, the flavor element 18 is attached to the surface of the wax layer 16. In an alternative embodiment, the strip 10 may not include a wax layer 16 and the flavor element may be attached directly to the substrate 12 opposite the oral care substance 14. As described below in more detail, in the illustrated embodiment the particles of film are attached to the wax layer when the wax is in a molten state, such that the particles are at least partially embedded in the wax when the wax has solidified. In one embodiment, the particles are stable at least up to the temperature of the molten wax, such that they do not melt completely when applied to the molten wax.

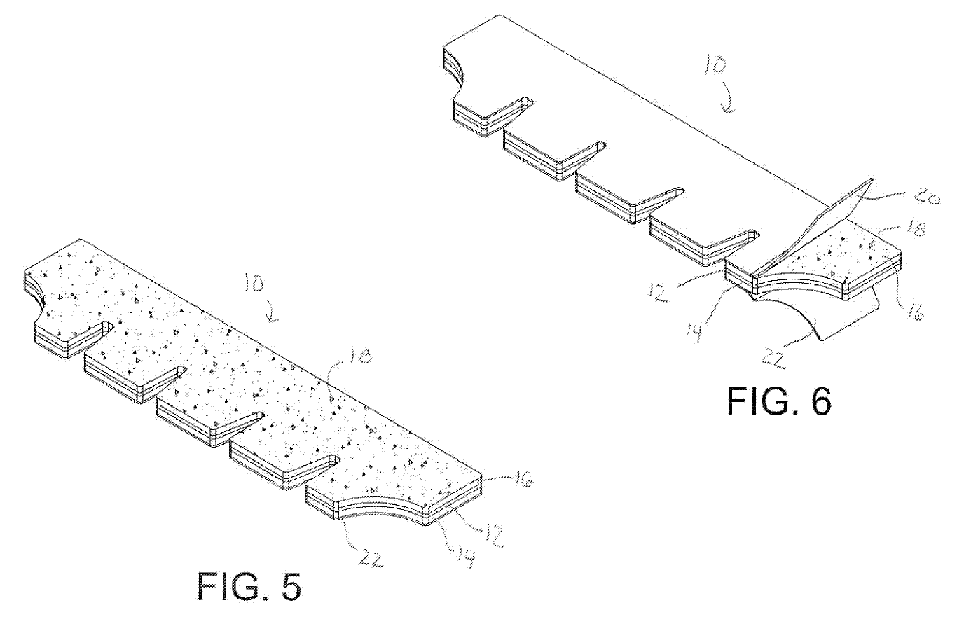

The carrier layers 20 and 22 are typically formed from the same thin, bendable material such as mylar or nylon. The first carrier 20 is such that it covers the flavor element 18 and wax layer 16 and may be peeled from the wax layer 16 when the wax is in solid form by a manufacturer or a consumer. The second carrier 22 is such that it covers the oral care substance 14 and may be releasably peeled from the oral care substance 14 by a manufacturer or a consumer. FIG. 6 shows the strip 10 as portions of the carriers 20 and 22 are peeled away. In one embodiment, shown in FIG. 5, the first carrier 20 is peeled away by the manufacturer before packaging, and the second carrier 22 remains on the strip 10 until removed by a consumer.

III. Manufacture

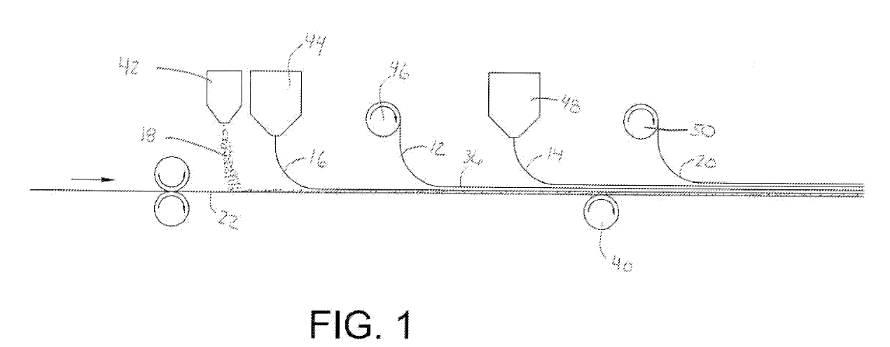

One embodiment of the method of manufacturing the present invention is shown schematically in FIG. 1. FIG. 1 schematically shows hoppers and rollers for dispensing the various components, however, various other devices can otherwise be used to hold and dispense the components. As shown, the various layers are added one on top of the other as they are moved by a number of rollers 40. The bottom or initial layer is the second carrier layer 22. The flavor element 18, shown in FIG. 1 as particles of flavored film, is then released from a hopper 42 onto the carrier 22. The wax layer 16, in a softened or molten form, is then extruded from a hopper 44 over the flavor element 18 and the carrier 22. The substrate 12 is dispensed from a roller 46 and layered on top of the wax 16, and the oral care substance 14 is dispensed from a hopper 48 onto the upper surface 36 of the substrate 12. Finally, the first carrier layer 20 is layered from a roller 50 on top of the oral care substance 14.

The flavor element 18 attaches to the wax layer as the wax is solidifying. More particularly, when the flavor element 18 is contacted by the softened wax, the flavor element 18 becomes at least partially embedded in the softened wax layer, such that it attaches to the wax layer as the wax 16 cools and solidifies. In an alternative embodiment, wherein the strip 10 does not include a wax layer, the flavor element 18 may be attached directly to the substrate 12 in a similar manner, for instance, the substrate 12 may be comprised of a polymer and the flavor element 18 may be placed on the polymer as it is in a molten form and attach to the polymer as it cures.

Once all of the layers are dispensed, the resulting layered product can then be cut into the strips 10 of a desired shape. In one embodiment, the second carrier 22 is peeled off the strips 10 by the manufacturer before the strips are packaged, such that the wax layer and flavor element are visible. The first carrier 20 is removed by the consumer just prior to inserting the strip into the mouth to expose the oral care substance 14. The strip 10 can then be inserted into the user's mouth such that the oral care substance 14 contacts the user's teeth, and the exposed flavor element provides flavor to the user's mouth. In the embodiment wherein the flavor element 18 includes particles of dissolvable, flavored film, the exposed film dissolves in the user's mouth when wiped with the tongue, lips, or otherwise passing saliva over the strip. In some cases, as portions of the wax are wiped away or removed, additional particles of flavor element 18 are revealed, thus providing longer lasting flavor for the user.

The above description is that of the current embodiment of the invention. Various alterations and changes can be made without departing from the spirit and broader aspects of the invention as defined in the appended claims, which are to be interpreted in accordance with the principles of patent law including the doctrine of equivalents. Any reference to claim elements in the singular, for example, using the articles “a,” “an,”“the” or “said,” is not to be construed as limiting the element to the singular.

The embodiments of the invention in which an exclusive property or privilege is claimed are defined as follows:

1. A flavored oral care product comprising:
a strip having a first surface and a second surface;
an oral care substance carried on said first surface; and
a flavor substance carried on said second surface, said flavor substance including a dissolvable film including one or more flavor ingredients.

2. The oral care product of claim 1 wherein said dissolvable film includes a plurality of particles.

3. The oral care product of claim 1 wherein said oral care substance is a tooth whitening gel.

4. The oral care product of claim 1 including a releasable strip attached over said oral care substance.

5. The oral care product of claim 1 wherein said dissolvable flavor substance is at least partially embedded in said second surface.

6. The oral care product of claim 1 wherein said second surface of said strip includes a wax layer, said flavor substance attached to said wax layer.

7. The oral care product of claim 6 including a carrier layer between said wax layer and said oral care substance.

8. A flavored oral care strip comprising:
a substrate having a first surface and a second surface opposite the first surface;
an oral care substance on said second surface of said substrate;
a wax layer on at least a portion of said first surface of said substrate; and
a plurality of dissolvable flavored particles at least partially embedded in said wax.

9. The flavored oral care product of claim 8 wherein said dissolvable flavored particles include a dissolvable binder and a flavor ingredient.

10. The flavored oral care product of claim 9 wherein said dissolvable binder is pullulan.

11. The flavored oral care product of claim 9 wherein said flavor ingredient includes at least one of peppermint, spearmint, wintergreen, cinnamon, berry and citrus flavors.

12. The flavored oral care product of claim 8 wherein said oral care substance is a tooth whitening agent.

13. A method for manufacturing a flavored oral care strip, comprising the steps of:
forming a wax into a strip;
placing a substrate on the strip of wax, the substrate having a first surface adjacent the wax and a second surface opposite the first surface;
placing a flavor substance onto a surface of the wax opposite the substrate; and
applying an oral care substance to the second surface of the substrate.

14. The method of claim 13 wherein the flavor elements include particles of a dissolvable, edible, flavored film.

15. The method of claim 14 wherein the film is comprised of at least pullulan and a flavor ingredient.

16. The method of claim 13 including applying a releasable carrier over the oral care substance.

17. The method of claim 13 including extruding the wax strip on a carrier, the carrier being releasable from the wax after the wax is solidified.

18. A method for manufacturing an oral care strip, comprising the steps of:
providing a strip of a releasable carrier;
applying a flavor element to the releasable carrier;
applying a wax layer over the flavor element; and
applying an oral care substance to the wax layer opposite the flavor element.

19. The method of claim 18 wherein the flavor element is applied to the wax when the wax is in a softened form such that the flavor element is at least partially embedded in the wax as the wax solidifies.

20. The method of claim 19 including providing a substrate between the wax layer and the oral care substance.

21. The method of claim 18 including applying a second releasable carrier over the oral care substance.

22. The method of claim 18 wherein the oral care substance is a tooth whitening gel.

23. The method of claim 18 wherein the flavor element is a plurality of particles of dissolvable, edible flavored film.
